(19) United States
(12) Patent Application Publication
(10) Pub. No.: US 2006/0259132 A1
(43) Pub. Date: Nov. 16, 2006

(54) Vascular stent for embolic protection

(75) Inventors: Darin G. Schaffer, Bloomington, IN (US); John A. Brumleve, Bloomington, IN (US); Kian Olsen, Bloomington, IN (US); Ram H. Paul JR., Bloomington, IN (US); Dharmendra Pal, Wilmington, MA (US); Fred T. Parker, Unionville, IN (US)

Correspondence Address: BRINKS HOFER GILSON & LIONE/CHICAGO/COOK, PO BOX 10395, CHICAGO, IL, 60610, US

(73) Assignee: Cook Incorporated, Bloomington, IN (US)

(21) Appl. No.: 11/414,807

(22) Filed: May 1, 2006

Related U.S. Application Data
(60) Provisional application No. 60/676,811, filed on May 2, 2005.

(51) Int. Cl. A61F 2/06

(52) U.S. Cl. 623/1.49

(57) ABSTRACT

A method and device to repair a stenosis in a blood vessel is provided. The medical device has a tubular member and a frame. The frame may be expanded or contracted while maintaining its generally cylindrical configuration. The medical device is retained in a contracted state inside an introducer sheath. The introducer sheath is guided through the stenosis such that a first end of the medical device is located distal the stenosis. The introducer sheath is retracted relative to the medical device, such that the first end of the stent expands to engage the blood vessel distal the stenosis. A mid-portion of the medical device engages the plaque of the stenosis trapping any emboli against the wall of the vessel. The second end expands to engage the blood vessel proximal to stenosis.

### CROSS-REFERENCE TO RELATED APPLICATIONS

This application claims the benefit of U.S. Provisional Application Ser. No. 60/676,811, filed on May 2, 2005, entitled “VASCULAR STENT FOR EMBOLIC PROTECTION,” the entire contents of which are incorporated herein by reference.

### BACKGROUND

1. Field of the Invention

The present invention generally relates to a system and method for repairing stenosed region of a blood vessel.

2. Description of Related Art

With the continuing advance of medical techniques, interventional procedures are more commonly being used to actively treat stenosis, occlusions, lesions, or other defects within a patient's blood vessels. Often the treated regions are in the coronary, carotid or even cerebral arteries. One procedure for treating an occluded or stenosed blood vessel is angioplasty. During angioplasty, an inflatable balloon is introduced into the occluded region. The balloon is inflated, pushing against the plaque or other material of the stenosed region and increasing the intralumenal diameter of the vessel. As the balloon presses against the material, portions of the material may inadvertently break free from the plaque deposit. These emboli may travel along the vessel and become trapped in a smaller blood vessel restricting blood flow to a vital organ, such as the brain.

Other methods for removing plaque or thrombus from arteries may include mechanical ablation, or non-contact ablation using light waves, sound waves, ultrasonics, or other radiation. Each of these methods are subject to the risk that some thrombogenic material may dislodge from the wall of the vessel and occlude smaller blood vessel. The occlusion may cause damage to the patient, including an ischemic stroke in the cerebral arteries.

To prevent the risk of damage from emboli, many devices have been used to restrict the flow of emboli downstream from the stenosed area. One method includes inserting a balloon that may be expanded to occlude the flow of blood through the artery downstream of the stenosed area. An aspirating catheter may be located between the balloon and stenosed area and used to remove emboli that may be caused by the treatment. However, because the balloon completely blocks blood flow through the vessel, the vessel may be occluded only for short periods of time, limiting use of the procedure.

As an alternative to occluding flow through the blood vessel, various filtering devices have been proposed. Such devices typically have elements that form legs or a mesh that would capture embolic material, but allow blood cells to flow between the elements. Capturing the emboli in the filter device prevents the material from being lodged downstream in a smaller blood vessel. The filter may then be removed along with the embolic material after the procedure has been performed and the risk from emboli has decreased.

In view of the above, there remains a need for an improved method and system for repairing a stenosed region of a blood vessel.

### SUMMARY

In satisfying the above need, as well as, overcoming the drawbacks and other limitations of the related art, the present invention provides an improved method and system for repairing a stenosed region of a blood vessel.

A stent is provided across a stenosed region of the blood vessel to trap emboli between the stent and the inner wall of the blood vessel. The stent has a tubular member and a frame, where the tubular member is attached to the frame and forms a lumen between a first and second end of the stent. The frame may be expanded or contracted to increase or decrease the diameter of the stent and lumen while maintaining its generally cylindrical configuration. For introduction into the blood vessel, the stent is retained in a contracted state inside an introducer sheath. The introducer sheath and stent are guided through the vasculature to the stenosis such that a first end of the stent is located distal the stenosis. The introducer sheath is retracted relative to the stent, such that the first end of the stent expands to engage an inner wall of the blood vessel distal the stenosis. A mid-portion of the stent expands to engage the stenosed area trapping any emboli against the wall of the vessel. As the introducer sheath is removed from around the second end of the stent, the second end expands to engage the inner wall of the blood vessel proximal to the stenosis, such that the stent, and more specifically the tubular member, extend along the entire length of the stenosis trapping emboli against the inner wall of the blood vessel.

In addition, a balloon catheter may be guided through the stent and dilated. Dilating an expandable portion of the balloon catheter forces the stent against the plaque of the stenosis thereby increasing the diameter of the stent and the corresponding region of the blood vessel. The balloon catheter is then removed from the blood vessel allowing blood to flow through the lumen between the first and second end of the stent.

The tubular member is preferably made of a bioimplantable material and more preferably is made of an extracellular matrix. The tubular member may be porous allowing blood cells to permeate the tubular member while retaining any emboli or plaque material against the wall of the blood vessel. In addition, the tubular member may also include a anti-thrombogenic substance, such as an anti-clotting drug, to dissolve any emboli that are formed.

The frame is made of structural members that may form a Z stent configuration or an interwoven configuration. The structural members may be biased to an expanded state or may be made of a shape memory alloy such that the temperature of the frame may be altered to bias the frame into an expanded state.

Further objects, features and advantages of this invention will become readily apparent to persons skilled in the art after a review of the following description, with reference to the drawings and claims that are appended to and form a part of this specification.

### BRIEF DESCRIPTION OF THE DRAWINGS

FIG. 1 is a front view of a human head generally depicting the path of the carotid arteries;

FIG. 2 is a sectional view of the branch of the blood vessel between the common carotid artery and the internal and external carotid arteries;

FIG. 3 is a sectional view of the blood vessel depicted in FIG. 2 showing a wire guide advanced through the stenosed region;

FIG. 4 is a sectional view of the blood vessel depicted in FIG. 2 further showing a section of the introducer sheath and stent advanced through the stenosed region;

FIG. 5 is a sectional view of the blood vessel depicted in FIG. 2 showing the first end of the stent deployed;

FIG. 6 is a side view of the stent of FIGS. 4 and 5 shown in a fully expanded state;

FIG. 7 is a sectional view of the blood vessel of FIG. 2 showing both ends of the stent deployed across the stenosis;

FIG. 8 is a sectional view of the blood vessel of FIG. 2 showing a balloon catheter advanced through the stent;

FIG. 9 is a sectional view of the blood vessel of FIG. 2 showing the balloon catheter fully dilated;

FIG. 10 is a sectional view of the fully deployed stent after the balloon catheter is allowed to contract; and

FIG. 11 is a sectional view of the blood vessel of FIG. 2 showing the fully deployed stent after the balloon catheter and wire guide are removed.

### DETAILED DESCRIPTION

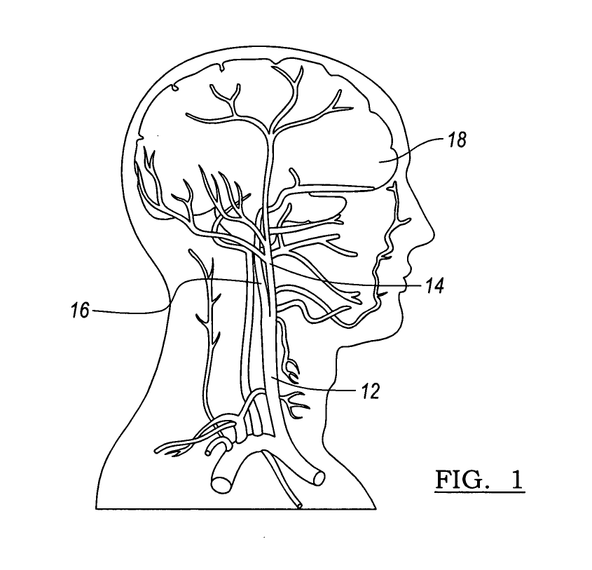

Referring now to FIG. 1, the path of the carotid arteries through the head of a patient is illustrated. The common carotid artery 12 travels from the aortic arch to the neck of the patient. The common carotid artery 12 splits into the external carotid artery 14 and the internal carotid artery 16. The external carotid artery 14 travels back along the neck line and provides blood to the back of the head and brain 18. The internal carotid artery 16 travels underneath the chin and up inside the head to provide blood to the eyes and the front of the brain 18. A branch is formed where the common carotid artery 12 splits into the external carotid artery 14 and internal carotid artery 16. Often plaque can collect at the branch causing stenosis inside the artery. This is particularly a problem with the carotid arteries that supply blood to the brain. If plaque breaks free forming emboli, the emboli may travel along the artery into the brain 18 blocking a small vessel in the brain 18 and causing an ischemic stroke.

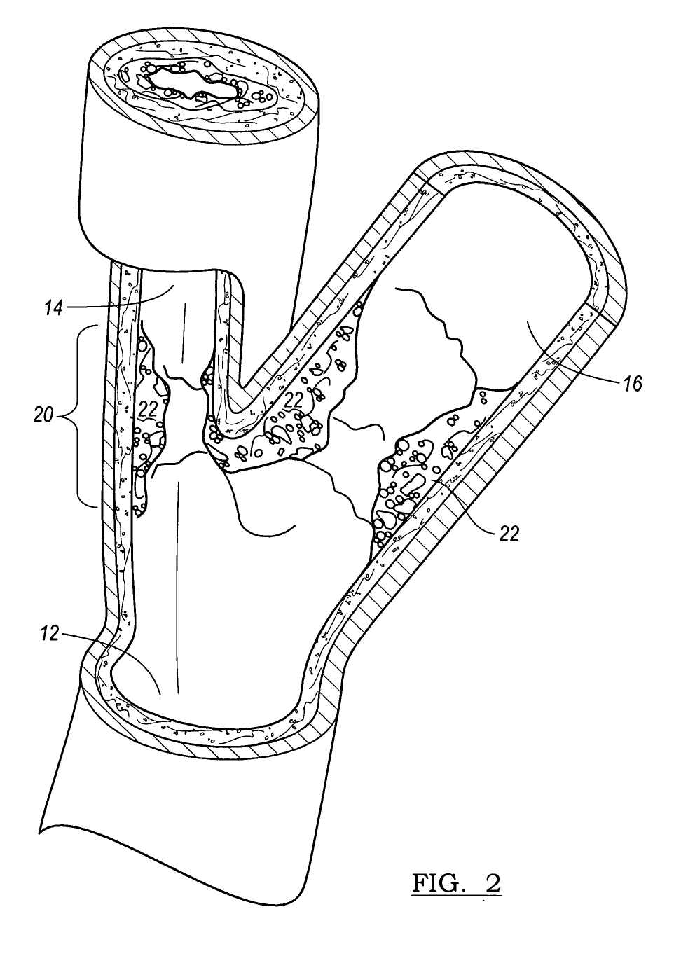

Now referring to FIG. 2, an enlarged cross-sectional view of the branch section 20 between the common carotid artery 12 and the internal and external carotid artery 14, 16 is provided in more detail. Embolic material is shown as plaque 22 on the side walls of the internal and external carotid arteries and at the apex of the branch. The plaque 22 forms a narrowing or stenosis 23 of the interal carotid artery 16. The flow in the external carotid artery 14 is shown to be about 50% to 60% occluded, while the occlusion in the internal carotid artery 16 is shown to be about 70% to 80%. For occlusions greater than 60%, a procedure will generally be performed to increase blood flow through the artery. One common concern is that pieces of the plaque 22 will break off during the procedure and block smaller vessels downstream of the stenosis.

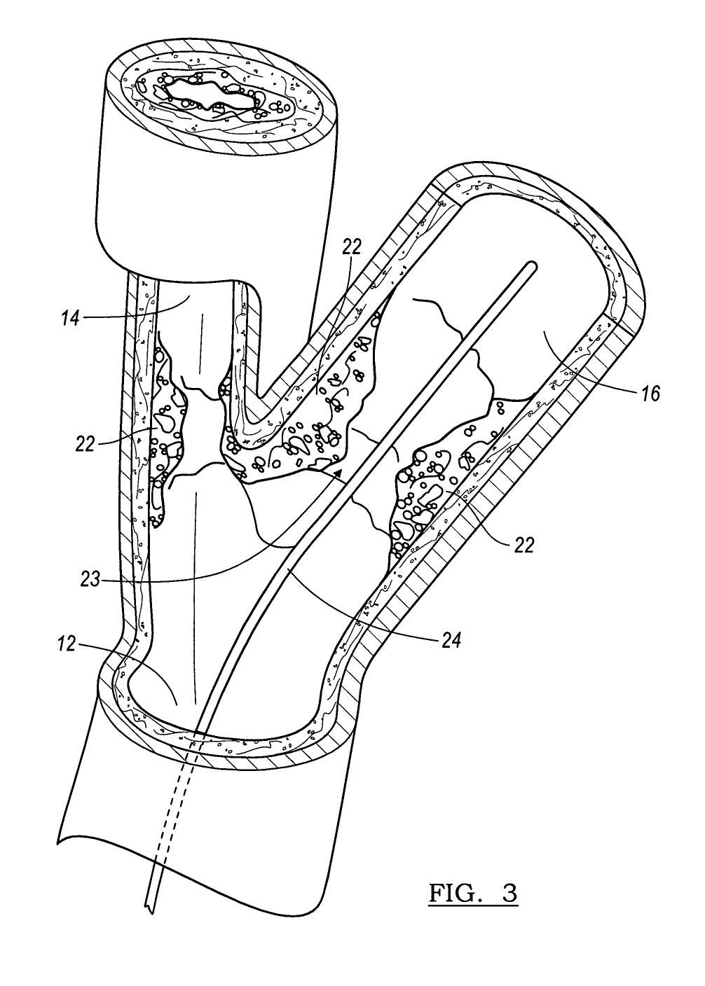

Now referring to FIG. 3, a wire guide 24 is inserted into the patient and advanced along the common carotid artery 12 through the stenosis 23 into the internal carotid artery 16. The wire guide 24 is typically less than 0.5 mm in diameter to pass through the stenosis without disturbing the plaque 22. The wire guide 24 is generally used to direct other devices to the region of interest. Accordingly, the devices generally include a lumen, such that the wire guide 24 is received through the lumen and the device is advanced over the wire guide 24 to the region of interest.

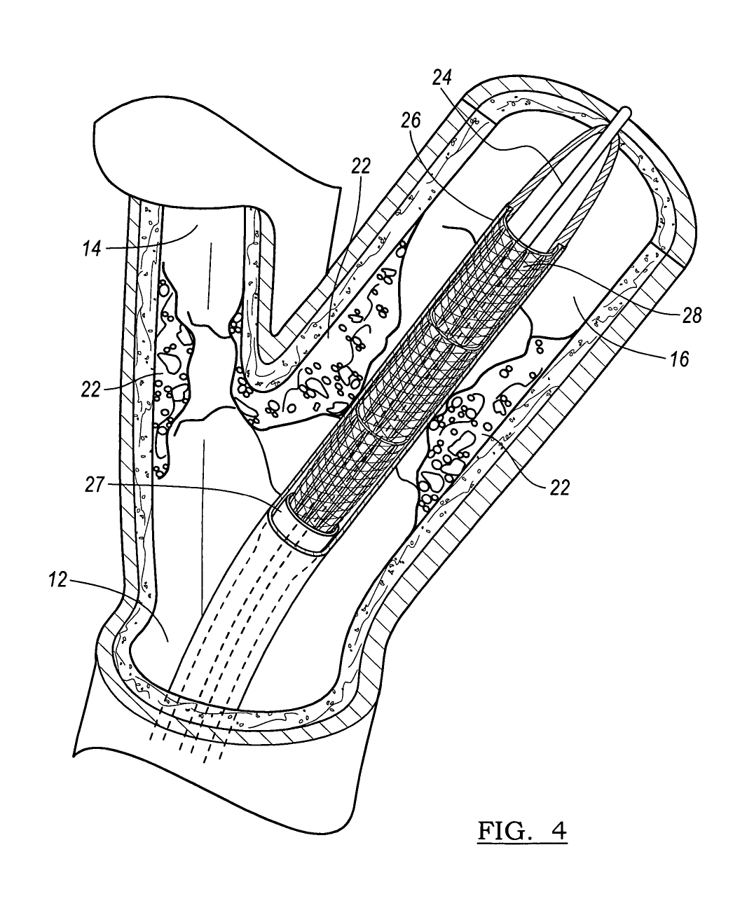

Now referring to FIG. 4, an introducer sheath 26 is advanced over the wire guide 24 through the stenosis 23 into the internal carotid artery 16. A stent 28 is located inside the introducer sheath 26. The stent 28 is compressed by the walls of the introducer sheath 26 to keep a tight, low profile as the sheath 26 and stent 28 are advanced through the stenosis 23, over the wire guide 24. An introducer 27 is located within the sheath 26 and behind the stent 28. The introducer 27 engages the distal end of the stent 28 and can be used to push the stent 28 distally relative to the sheath 26. As the sheath 26 is passed through the stenosis 23, most or virtually all of the blood flow between the common carotid artery 12 and the internal carotid artery 16 may blocked due to the stenosis 23. If portions of the plaque 22 are broken off, any emboli tend to remain stagnant since there is little or no blood flow.

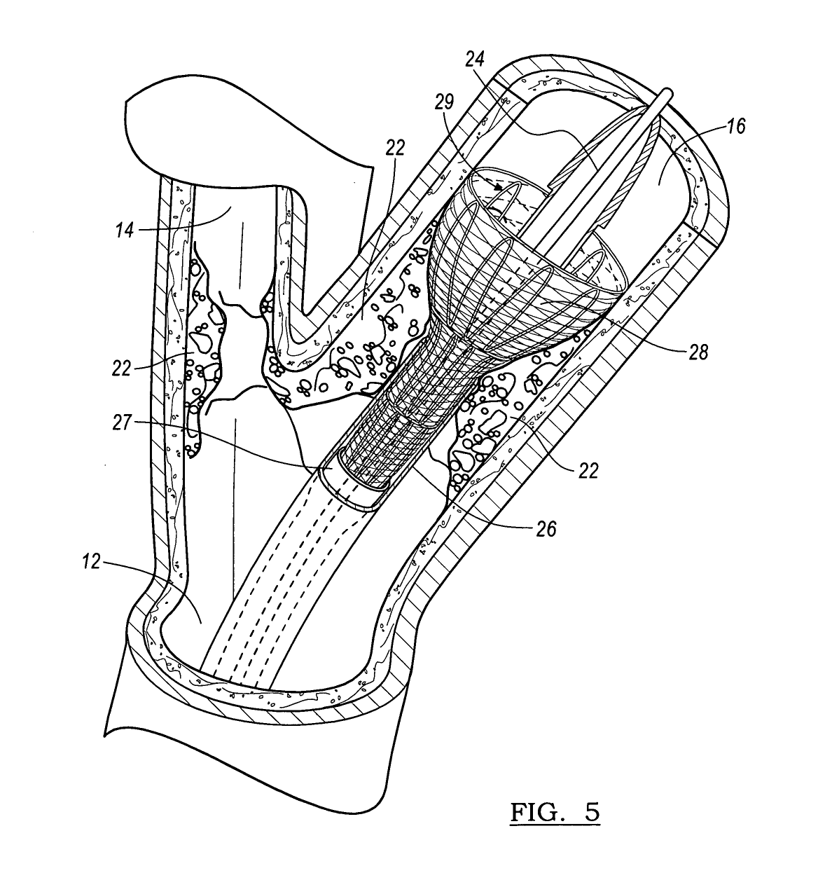

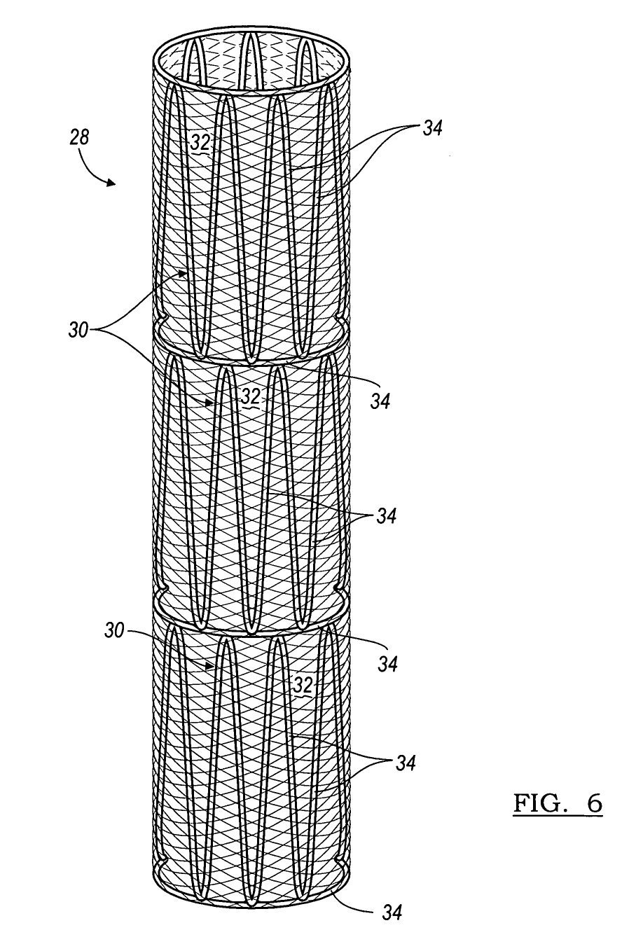

Now referring to FIG. 5, as a first end 29 of the stent 28 is located distal to the stenosis 23. The sheath 26 is retracted back over the stent 28 such that the first end 29 of the stent 28 is free and expands against the inside wall of the internal carotid artery 16 distal the stenosis 23. The stent 28 includes a frame 30 and a tubular member 32, as shown in FIG. 6. The frame 30 may be made of a plurality of structural members 34 configured to have an expanded or contracted state. As such, the structural members 34 may form a diagonal Z configuration to expand and contract while maintaining a generally cylindrical geometry. The frame 30 may be made of stainless steel and biased to an expanded state. Alternatively, the frame 30 may comprise a shape memory material such as Nitinol, and the temperature of the frame may be altered biasing the structural members to the expanded state.

For example, a fluid may be provided through the sheath 26 to alter the state of the shape memory material thereby biasing the frame to the expanded state. The tubular member 32 is attached to the frame 30 and configured to extend along the length of the stenosis 23. The tubular member 32 may be made of synthetic biocompatible material, such as Dacron, Thoralon, or expanded polytetrafluoroethylene (ePFTE) material. While synthetic biocompatible materials can be used to fabricate the coverings for stents, a naturally occurring material biomaterial, such as collagen, is highly desirable. Particularly desirable is a specially derived collagen material known as an extracellular matrix (ECM), such as small intestinal submucosa (SIS). Besides SIS, examples of ECM's include pericardium, stomach submucosa, liver basement membrane, urinary bladder submucosa, tissue mucosa, and dura mater. Further, the tubular member 32 may be made of an extracellular matrix, such that the tubular member may be absorbed into the inner wall of the blood vessel over a period of time. Accordingly, the tubular member 32 is attached to and extends along the outside of the frame 30.

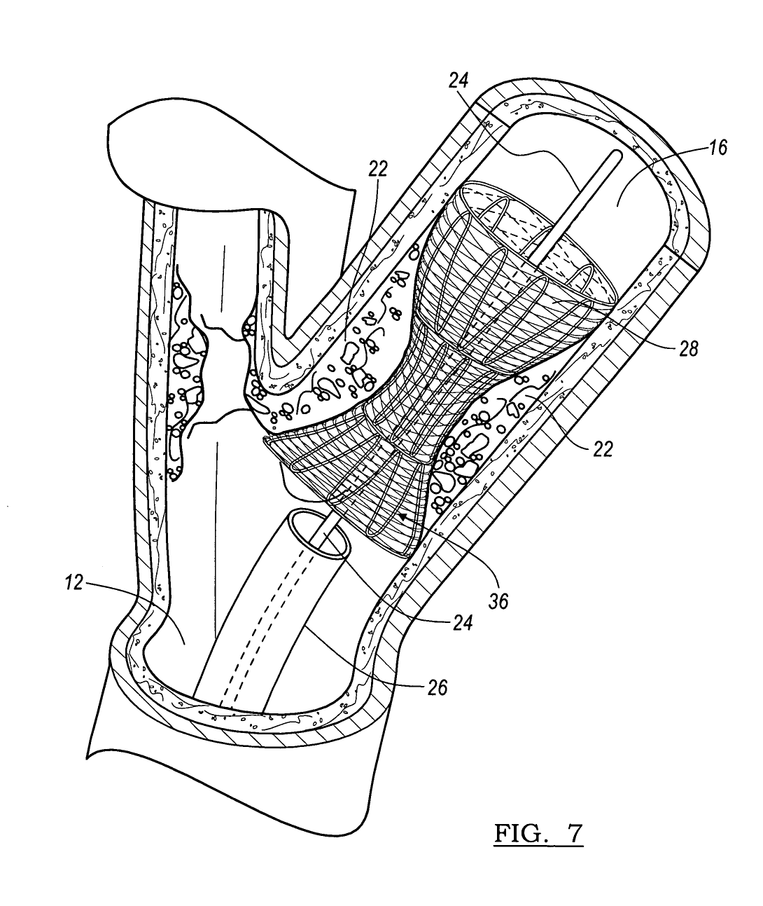

As the introducer sheath 26 is retracted further, as shown in FIG. 7, a mid-portion of the stent 28 may expand against the plaque 22 trapping any emboli against the walls of the blood vessel. In addition, a second end 36 of the stent 28 is expanded to engage the inner wall of the blood vessel proximal the stenosis 23, such that the tubular member 32 extents along the entire length of the stenosis 23. Further, the tubular member 32 has pores allowing blood cells to pass thorugh the surface of the tubular member 32, while emboli are restrained by the tubular member 32. Accordingly, the tubular member 32 would be permeable to objects less than about 30 microns. The tubular member 32 may also be treated with an anti-thrombogenic substance to promote dissolution of any emboli trapped by the tubular member 32.

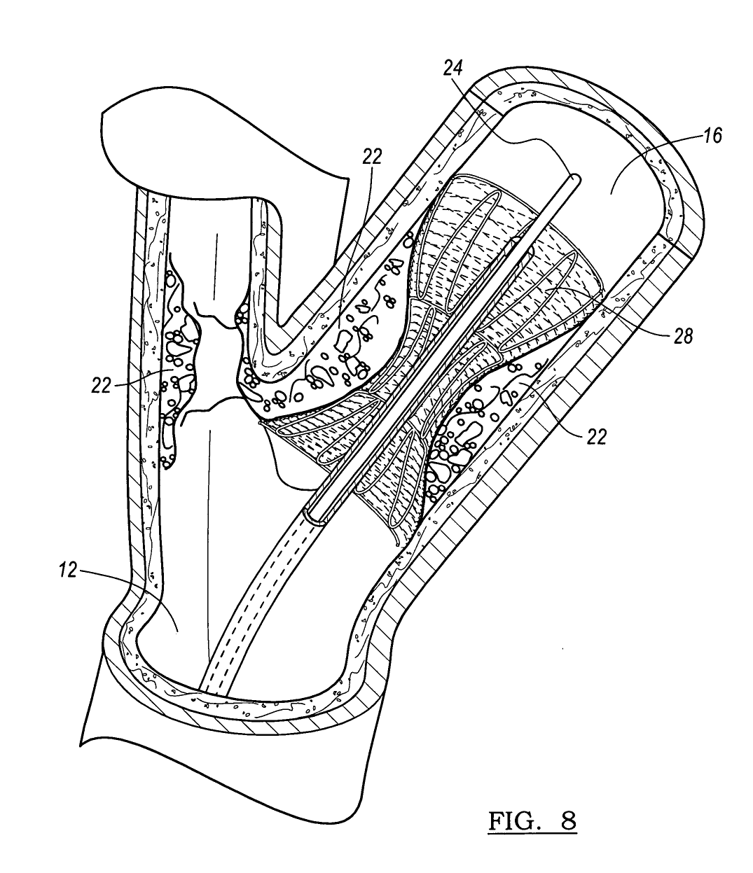

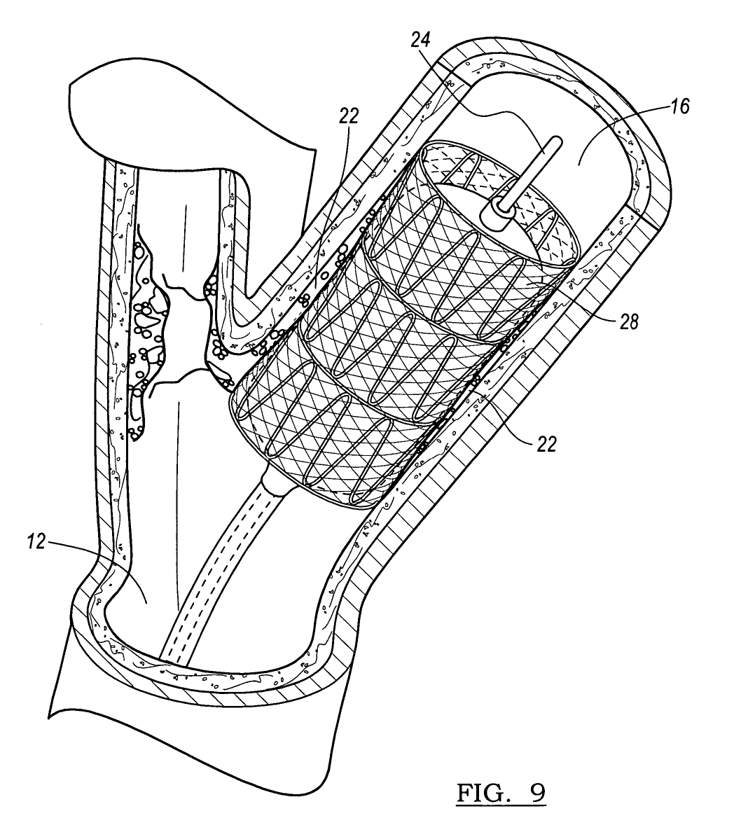

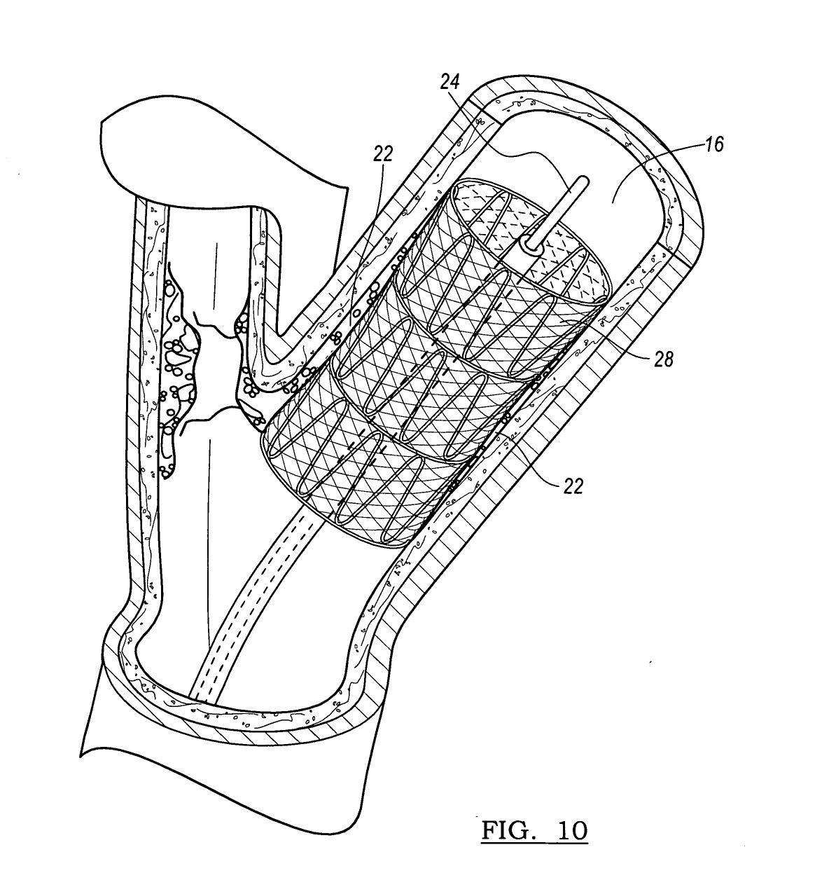

After the stent is deployed and free from the sheath 26, the sheath 26 may then be fully removed from the patient. Then a balloon catheter 32 may be advanced over the wire guide 24 through the stent 28, as shown in FIG. 8. The balloon catheter 32 has an inner lumen to allow advancement over the wire guide 24 and an outer lumen allowing fluid to be pumped into an expandable portion of the balloon catheter 32. As shown in FIG. 9, the expandable portion of the balloon catheter 32 is located within the stent 28 and dilated. With the balloon catheter 32 fully dilated, the plaque material 22 is compressed against the walls of the blood vessel further expanding the lumen through the stent 28. The balloon catheter 32 is then allowed to contract, as shown in FIG. 10. The frame of the stent 28 continues to support the stent 28 against the plaque material 22, also trapping any emboli between the stent 28 and the inner wall of the blood vessel.

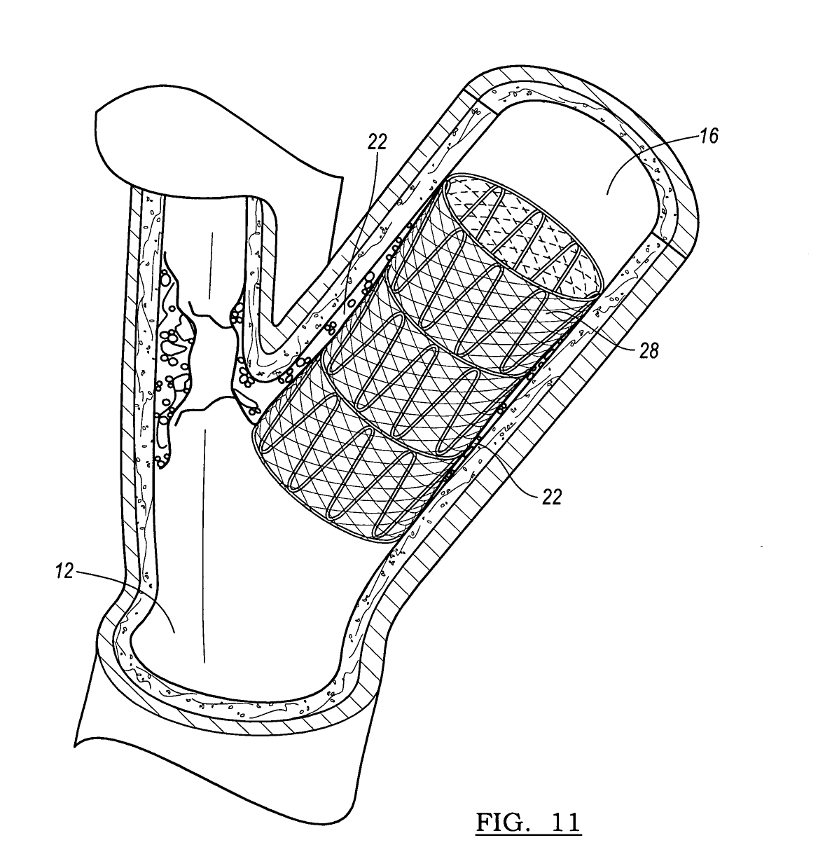

Now referring to FIG. 11, after the balloon catheter 32 is removed, the stent 28 remains in place to trap emboli against the wall of the internal carotid artery 16. Over time, the stent 28 being made of biocompatible material will be absorbed into the wall of the internal carotid artery 16 permanently trapping the plaque material 22.

As a person skilled in the art will readily appreciate, the above description is meant as an illustration of the principles of this invention. This description is not intended to limit the scope or application of this invention in that the invention is susceptible to modification, variation and change, without departing from spirit of this invention, as defined in the following claims.

I/We claim:

1. A method for treating a stenosis in a blood vessel, the method comprising:
providing a stent having an expanded state and a contracted state, the stent comprising a frame and a tubular member, a lumen extending between first and second ends of the tubular member, the tubular member being permeable to blood and being configured to constrain emboli between the tubular member and the blood vessel, the tubular member being sized to run along the entire length of the stenosis;
delivering the stent to the vessel proximate the stenosis;
expanding the first end of the stent to engage an inner wall of the blood vessel distal the stenosis;
expanding a mid-portion of the stent to engage the stenosis; and
expanding a second end of the stent to engage the inner wall of the blood vessel proximal the stenosis.

2. The method according to claim 1, wherein the step of delivering the stent is performed such that substantially all blood flow through the blood vessel is blocked.

3. The method according to claim 1, further comprising:
providing a balloon catheter including an expandable portion;
guiding the balloon catheter through the stent after the step of expanding the first end of the stent to engage an inner wall of the blood vessel;
dilating the expandable portion to force the stent against the stenosis thereby increasing the diameter of the lumen; and
removing the balloon catheter thereby allowing blood flow through the lumen.

4. The method according to claim 1, wherein the frame includes a plurality of expandable members attached to the tubular member.

5. The method according to claim 1, wherein the tubular member is comprised of an extracellular matrix.

6. The method according to claim 5, wherein the tubular member is comprised of a SIS material.

7. The method according to claim 1, wherein the tubular member is comprised of a synthetic biocompatible material.

8. The method according to claim 1, wherein the tubular member is permeable to objects less than 30 microns.

9. The method according to claim 1, wherein the frame is biased to the expanded state.

10. The method according to claim 1, wherein the frame comprises a shape memory material, and wherein the temperature of the stent is altered to bias the frame to the expanded state.

11. The method according to claim 1, wherein the tubular member includes an anti-thrombogenic substance.

12. A method for treating a stenosis in a blood vessel, the method comprising:
providing a stent having a tubular member and a frame, the tubular member is attached to the frame and has a lumen located between a first and second end of the stent, the frame being self expandable to define an expanded state and a contracted state of the stent, further wherein the tubular member comprises an extracellular matrix; the tubular member being permeable to blood and being configured to constrain emboli between the tubular member and the blood vessel, the tubular member being sized to run along the entire length of the stenosis;
delivering the stent to the vessel proximate the stenosis;
expanding the first end of the stent such that the extracellular matrix engages an inner wall of the blood vessel distal the stenosis;
expanding a mid-portion of the stent to engage the stenosis;
expanding a second end of the stent such that the extracellular matrix engages the inner wall of the blood vessel proximal the stenosis.

13. The method according to claim 12, wherein the step of delivering the stent is performed such that substantially all blood flow through the blood vessel is blocked.

14. The method according to claim 12, further comprising:
providing a balloon catheter including an expandable portion;
guiding the balloon catheter through the stent;
dilating the expandable portion to force the stent against the stenosis thereby increasing the diameter of the lumen;
removing the balloon catheter allowing blood flow through the lumen.

15. The method according to claim 12, wherein the tubular member extends along the entire length of the stenosis.

16. The method according to claim 12, wherein the tubular member is comprised of a SIS material.

17. The method according to claim 12, wherein the tubular member is permeable to objects less than 30 microns.

18. The method according to claim 12, wherein the tubular member includes an anti-thrombogenic substance.

19. A medical device for treating a stenosis in a blood vessel, the medical device comprising:
a frame being expandable to define an expanded and contracted state, the frame being biased to the expanded state;
a tubular member attached along a length of the frame and forming a lumen between first and second ends of the frame, the tubular member being configured with the frame in the expanded state to engage the blood vessel at the first and second end, the tubular member comprising an extracellular matrix, the extracellular matrix being permeable to blood and being configured to constrain emboli between the tubular member and the blood vessel, the tubular member being sized to run along the entire length of the stenosis.

20. The medical device according to claim 19, wherein the tubular member is comprised of a SIS material.

21. The medical device according to claim 19, wherein the tubular member is permeable to objects less than 30 microns.

22. The medical device according to claim 19, wherein the tubular member includes an anti-thrombogenic substance.

23. The medical device according to claim 19, wherein the frame comprises a shape memory material, and wherein the temperature of the stent is altered to bias the frame to the expanded state.
